SEQUENCE LISTING (6 sequences)

SEQ ID NO: 1 - DNA, 22 bp, nematoda
acagtcttatcgggacctcaaa

SEQ ID NO: 2 - DNA, 22 bp, NEMATODA
agagcccatcaacactacaacc

SEQ ID NO: 3 - DNA, 22 bp, NEMATODA
caccttggtgctacttcttgct

SEQ ID NO: 4 - DNA, 21 bp, NEMATODA
gggtagactggctcgttcctt

SEQ ID NO: 5 - DNA, 22 bp, NEMATODA
accgagcgtggttactctttca

SEQ ID NO: 6 - DNA, 21 bp, NEMATODA
tccgacggtgatgacttgtcc

(19) United States
(12) Patent Application Publication
(10) Pub. No.: US 2016/0369281 A1
(43) Pub. Date: Dec. 22, 2016

(54) Target point, preparation and method for treating human ADSL deficiency

(71) Applicant: Geneheal Biotechnology Co., Ltd., Guangzhou City (CN)

(72) Inventor: Xionglei HE, Guangzhou City (CN)

(21) Appl. No.: 14/741,158

(22) Filed: Jun. 16, 2015

(51) Int. Cl. C12N 15/113

(52) U.S. Cl. CPC C12N 15/1137; C12N 2310/14; C12N 2320/30

(57) ABSTRACT

The present invention discloses target point, preparation and method for treating human ADSL deficiency. The present invention established an ADSL deficiency retrieval model in nematode through RNA interference technique, and found that the sole RNA interference with PAICS gene could increase the expression of ADSL gene. Based on the conclusion of nematode model study, it can be foreseen that through interference with PAICS gene expression in ADSL deficient patient, accumulation of SAICAR in patient body fluid will be decreased and thereby reducing damage to cells, achieving the object of treating human ADSL deficiency or relieving symptoms.

### BACKGROUND OF THE INVENTION

Technical Field

The invention relates to a drug target of a genetic malady, and preparation and treating method thereof.

Background Art

Purine anabolism is ubiquitous and important biological metabolism producing AMP and GMP which provides not only raw materials for biosynthesis of DNA and RNA in vivo, but also purine bases necessary for synthesis of key coenzymes (NAD, NADP, FAD and CoA), signal molecules (e.g. cAMP) and the important energy molecule ATP in vivo. It can be seen that purine anabolism occupied a central position in the whole metabolic network. Purine synthesis involves two synthetic pathways including de novo purine synthesis and salvage pathway.

Adenylosuccinatelyase deficiency (ADSL) is a metabolic disease which causes deletion and disorder during de novo adenine synthesis and in the metabolic pathway of purine nucleotides. The main cause of the disease is that the mutation or deletion of adenylosuccinatelyase occurring in patient's body leads to excessive accumulation of the enzyme substrate SAICAR in cells, which cannot be timely removed [Jaeken J, Van den Berghe G. (1984). An infantile autistic syndrome characterized by the presence of succinylpurines in body fluids. Lancet 8411: 1058-1061]. The accumulation of the metabolite was detected for the first time by Jaeken and Van den Berghe in the body fluids of several patients suffering from bradykinesia and autism. Symptoms such as severe dysplasia, bradykinesia, eyes glazed over, seizures, autism usually appear in patients with adenylosuccinatelyase deficiency [Spiegel, E. K., Colman, R. F., and Patterson, D. (2006).

Adenylosuccinatelyase deficiency. Mol Genet Metab 89, 19-31. Clamadieu, C., Cottin, X., Rousselle, C., and Claris, O. (2008). Adenylosuccinate lyase deficiency: an unusual cause of neonatal seizure. Arch Pediatr 15, 135-138. Castro, M., Perez-Cerda, C., Merinero, B., Garcia, M. J., Bernar, J., Gil Nagel, A., Tones, J., Bermudez, M., Garavito, P., Marie, S., et al. (2002). Screening for adenylosuccinate lyase deficiency: clinical, biochemical and molecular findings in four patients. Neuropediatrics 33, 186-189. Jurecka, A., Zikanova, M., Tylki-Szymanska, A., Krijt, J., Bogdanska, A., Gradowska, W., Mullerova, K., Sykut-Cegielska, J., Kmoch, S., and Pronicka, E. (2008b). Clinical, biochemical and molecular findings in seven Polish patients with adenylosuccinate lyase deficiency. Mol Genet Metab 94, 435-442].

Adenylosuccinate lyase is mainly involved in two reactions which catalytically cleave SAICAR into AICAR and S-AMP into AMP respectively in the metabolic pathway of de novo adenine synthesis [Spiegel, E. K., Colman, R. F., and Patterson, D. (2006). Adenylosuccinate lyase deficiency. Mol Genet Metab 89, 19-31. Clamadieu, C., Cottin, X., Rousselle, C., and Claris, O. (2008). Adenylosuccinate lyase deficiency: an unusual cause of neonatal seizure. Arch Pediatr 15, 135-138. Castro, M., Perez-Cerda, C., Merinero, B., Garcia, M. J., Bernar, J., Gil Nagel, A., Tones, J., Bermudez, M., Garavito, P., Marie, S., et al. (2002). Screening for adenylosuccinate lyase deficiency: clinical, biochemical and molecular findings in four patients. Neuropediatrics 33, 186-189]. Due to the mutation or deletion of ADSLlyase in patients with adenylosuccinate lyase deficiency, harmful metabolites SAICAR cannot be timely removed, which generally leads to severe neurological and physiological symptoms, such as seizures, cerebral hypoplasia, sluggish in motion, and so forth. [Ciardo, F., Salerno, C., and Curatolo, P. (2001). Neurologic aspects of adenylosuccinate lyase deficiency. J Child Neurol 16, 301-308. Gitiaux, C., Ceballos-Picot, I., Marie, S., Valayannopoulos, V., Rio, M., Verrieres, S., Benoist, J. F., Vincent, M. F., Desguerre, I., and Bahi-Buisson, N. (2009). Misleading behavioural phenotype with adenylosuccinate lyase deficiency. Eur J Hum Genet 17, 133-136. Mierzewska, H., Schmidt-Sidor, B., Jurkiewicz, E., Bogdanska, A., Kusmierska, K., and Stepien, T. (2009). Severe encephalopathy with brain atrophy and hypomyelination due to adenylosuccinate lyase deficiency—MRI, clinical, biochemical and neuropathological findings of Polish patients. Folia Neuropathol 47, 314-320]. In general, large amount of intermediate metabolites SAICAr and S-Ado (SAICAr is a dephosphorylated product of SAICAr, and S-Ado is a dephosphorylated product of S-AMP) are accumulated in cerebrospinal and body fluid [Spiegel, E. K., Colman, R. F., and Patterson, D. (2006). Adenylosuccinate lyase deficiency. Mol Genet Metab 89, 19-31. Mierzewska, H., Schmidt-Sidor, B., Jurkiewicz, E., Bogdanska, A., Kusmierska, K., and Stepien, T. (2009). Severe encephalopathy with brain atrophy and hypomyelination due to adenylosuccinate lyase deficiency—MRI, clinical, biochemical and neuropathological findings of Polish patients. Folia Neuropathol 47, 314-320]. Van den Berghe et al., found that there is certain correlation between the S-do/SAICAr ratio in body fluid and the severity of the disease in patients. [Van den Bergh F, Vincent M F, Jaeken J, Van den Berghe G. (1993). Residual adenylosuccinase activities in fibroblasts of adenylosuccinase-deficient children: parallel deficiency with adenylosuccinate and succinyl-AICAR in profoundly retarded patients and non-parallel deficiency in a mildly retarded girl, J. Inherit. Metab. Dis. 16 (2) 415-424].

The ADSL gene presents as an essential gene in human body, and complete deletion of it will lead to congenital lethal symptoms. The activity of ADSLlyase is not entirely lost in all ADSL-deficient patients [Van den Bergh F, Vincent M F, Jaeken J, Van den Berghe G. (1993). Residual adenylosuccinase activities in fibroblasts of adenylosuccinase-deficient children: parallel deficiency with adenylosuccinate and succinyl-AICAR in profoundly retarded patients and non-parallel deficiency in a mildly retarded girl, J. Inherit. Metab. Dis. 16 (2) 415-424. Van den Bergh F, Vincent M F, Jaeken J, Van den Berghe G., (1993). Functional studies in fibroblasts of adenylosuccinase-deficient children, J. Inherit. Metab. Dis. 16 (2) 425-434]. 38 mutation sites of ADSL gene have been found so far [Spiegel, E. K., Colman, R. F., and Patterson, D. (2006). Adenylosuccinate lyase deficiency. Mol Genet Metab 89, 19-31], and more will be found as the number of patients diagnosed increases. These mutation sites have decreased the activity of ADSLlyase in patient to a variable extent, leading to the accumulation of harmful metabolites SAICAr in body.

Diagnoses of ADSL deficiency in patients are clinically through the determination of the content of the purine metabolites in the cerebrospinal fluid and body fluid, in particular, the accumulation of harmful metabolites SAICAr. At the earliest, people used Bratton-Marshall assay [Laikind P K, Seegmiller J E, Gruber H E, (1986). Detection of 5′-phosphoribosyl-4-(N-succinylcarboxamide)-5-aminoimidazole in urine by use of the Bratton-Marshall reaction: identification of patients deficient in adenylosuccinate lyase activity, Anal. Biochem. 156. (1) 81-90] involving the method for analysis of diazotized amine. However, this method was abandoned gradually as it may yield false positive results after administration of the related drug. Determination of the content of SAICAr and S-Ado in the cerebrospinal fluid and body fluid through HPLC is the most common way in the present day [Marie S, Flipsen J W, Duran M, Poll-The B T, Beemer F A, Bosschaart A N, Vincent M F, Van den Berghe G, (2000a). Prenatal diagnosis in adenylosuccinate lyase deficiency. PrenatDiagn 20, 33-36. Domkin V D, Lazebnik T A, Roudneff A, Smirnov M N, (1995). A new diagnostic technique for adenylosuccinate lyase deficiency. J Inherit Metab Dis 18, 291-294]. There is no clinically effective therapeutic regimen for curing ADSL deficiency at present.

Ten enzymatic reactions are needed to synthesize IMP from PRPP during the de novo purine synthesis [Hartman, S. C. & Buchanan, J. M. (1959). Biosynthesis of the purines. XXVI. The identification of the formyl donors of the transformylation reactions. J. Biol. Chem. 234, 1812-1816. Lukens, L. N. & Buchanan, J. M. (1959). Biosynthesis of the purines. XXIV. The enzymatic synthesis of 5-amino-1-ribosyl-4-imidazolecarboxylic acid 5 ‘-phosphate from 5-amino-1-ribosylimidazole 5’-phosphate and carbon dioxide. J. Biol. Chem. 234, 1799-1805]. The study found that the growth of cancer cells mainly (some cancer cells totally) rely on the nucleotide produced by the pathway of de novo purine synthesis, while normal cells tend to use nucleotide produced by salvage pathway [Jackson, R. & Harkrader, R. (1981). Nucleosides and Cancer Treatment. Academic Press, Sydney]. It is found that many kinds of cancer, such as about 30% of T-cell acute lymphoblastic leukemia lack alternative pathways for synthesis of adenosine, and need to completely rely on the pathway of de novo purine synthesis [Batova, A., Diccianni, M. B., Omura-Minamisawa, M., Yu, J., Carrera, C. J., Bridgeman, L. J. et al. (1999). Use of alanosine as a methylthioadenosinephosphorylase selective therapy for T-cell acute lymphoblastic leukemia in vitro.

Cancer Res. 59, 1492-1497]. Phosphoribosylaminoimidazolesuccinocarboxamidesynthetase/phosphoribosylaminoimidazole carboxylase, namely PAICS, is an important bifunctional enzyme in the pathway of de novo purine synthesis, having the functions of SAICAR synthetase (4-(N-succinylcarboxamide)-5-aminoimidazole ribonucleotide synthetase, SAICARs) and AIR carboxylase (5-aminoimidazole ribonucleotide carboxylase, AIRc) to catalyze the steps 6 and 7 in the pathway for de novo purine synthesis. Functional complementation of Escherichia coli pur mutants was first used to clone avian PAICS gene in 1990, and proved that SAICARs and AIRc are located at the N-terminal and C-terminal of PAICS, respectively [Chen, Z. D., Dixon, J. E. & Zalkin, H. (1990). Cloning of a chicken liver cDNA encoding 5-aminoimidazole ribonucleotide carboxylase and 5-aminoimidazole-4-N-succinocarboxamide ribonucleotide synthetase by functional complementation of Escherichia coli pur mutants. Proc. Natl Acad. Sci. USA, 87, 3097-3101]. In 2007, it was first reported that human PAICS presents in the form of octamer, wherein flower-like octameric structure consists of octamerizing AIRc as core and four dimerizing SAICARs as petal. In addition, the study also found that four mutually independent channel systems present in the octamer of PAICS. Each channel system connects with two AIRcs and two SAICARs active sites [Li S X., Tong Y P., Xie X C., Wang Q H., Zhou H N., Han Y, et al. (2007). Octameric structure of the human bifunctional enzyme PAICS in purine biosynthesis. J Mol Biol. 366(5):1603-1614]. In 2009, it was reported that PAICS plays a crucial role in vertebrate embryonic development [Ng A., Uribe R A., Yieh L., Nuckels R., Gross J M. (2009). Zebrafish mutations in gart and paics identify crucial roles for de novo purine synthesis in vertebrate pigmentation and ocular development. Development. 136(15):2601-2611]. Yet, it has not found that PAICS effects in decreasing the synthesis or accumulation of SAICAR.

### BRIEF SUMMARY OF THE INVENTION

One object of the invention is to provide a therapeutic target for treating human ADSL deficiency.

Another object of the invention is to provide a preparation for treating human ADSL deficiency.

Yet another object of the invention is to provide a method for treating human ADSL deficiency.

The present invention established an ADSL deficiency retrieval model in nematode through RNA interference technique. The efficiency of RNA interference in nematode can be well evaluated through RT-PCR. The study found that ADSL gene expression quantity will increase after the PAICS gene is solely treated with RNA interference.

The results of phenotype observation on the growth and development of nematode indicated that the sole RNA interference with ADSL gene in nematode will lead to severe deficient phenotype, such as growth and development retardation, sluggish in motion, decrease of spawning amount and so on. The growth rate of nematode is about 73.94% of the negative control after deletion of ADSLlyase. And the sole RNA interference with PAICS gene substantially has no effect on the growth and development of nematode, which is relevant with the fact that PAICS gene is a dispensable one. In contrast, after simultaneous RNA interference with PAICS in nematode, severe phenotype of ADSL deficiency can be well retrieved, and the growth and development of nematode also tends to be normal, reaching 92.4% of the negative control.

The analysis of adenine intermediate metabolites through LC-MS showed that large amount of intermediate metabolites SAICAr were accumulated in nematode body when ADSL was deleted. According to KEGG metabolic network, the intermediate metabolites SAICAR of de novo adenine synthesis cannot be removed efficiently. SAICAR presents largely in dephosphorylated form SAICAr, which is consistent with clinical diagnosis of accumulation of large amount of SAICAr in the body fluid of ADSL deficient patient. It proved that if SAICAr was substantially accumulated in the body and cannot be removed efficiently, it will result in deleterious effects on cells. While if PAICS gene (encoding the synthetase of SAICAR) and ADSL gene are both treated with RNA interference, the phenotypes of growth and development disorder caused by deletion of the essential gene ADSL can be retrieved. The synthesis of SAICAR can be decreased effectively by simultaneous RNA interference with PAICS gene expression, thereby retrieving the phenotype with ADSL deficiency. This hypothesis was verified when metabolites of RNA double interfered nematode were analyzed, as SAICAR was no longer accumulated in nematode (accumulation of SAICAR and SAICAr cannot be detected by LC-MS), and the growth and development of which tended to be normal. From the metabolite perspective, this study analyzed the toxic action of harmful metabolites accumulation in cell and the practicability of ADSL deficiency retrieval model established in nematode.

Based on the conclusion of nematode model study, it can be foreseen that through interfering with PAICS gene expression in ADSL deficient patients, accumulation of SAICAR in patient body fluid will be decreased, and thereby reducing damage to cells, achieving the object of treating human ADSL deficiency or relieving symptoms thereof.

### BRIEF DESCRIPTION OF THE SEVERAL VIEWS OF THE DRAWINGS

FIG. 1 shows a total RNA electrophoretogram of the nematode in F3 generation;

FIG. 2 shows the relative expression of PAICS gene and ADSL gene in different treatment groups;

FIG. 3 shows the relative growth rate of the nematode after RNA interference (0 day, 3 days, 6 days);

FIG. 4 show the relative increase in length of the nematode after RNA interference.

### DETAILED DESCRIPTION OF THE INVENTION

Target point for treating human ADSL deficiency, which is at least one selected from:

PAICS gene;
mRNA of the PAICS gene;
and PAICS protein.

Preparation for treating human ADSL deficiency, comprising at least one selected from: reagents and/or oligonucleotides that may interfere with the PAICS gene expression;

reagents and/or oligonucleotides that may interfere with the normal function of the mRNA of PAICS gene;
reagents that may inactivate the specificity of the PAICS protein.

Reagents that may interfere with the PAICS gene expression are selected from the group comprising polypeptide hormone, enzyme, interferon, interleukin, colony stimulating factor, a nucleotide sequence that may be hybridized with PAICS gene or a modified nucleotide sequence, recombinant protein, etc.

Oligonucleotides that may interfere with the PAICS gene expression or may interfere with the normal function of the mRNA of PAICS gene at least comprise a nucleotide sequence that may be hybridized with the cDNA sequence of the human PAICS gene (GenBank ID 30582814).

As general knowledge in the art, in order to ensure the stability and specificity of the hybridization, the length of the nucleotide sequence which is hybridized with PAICS or its cDNA, mRNA, or is modified is not less than 5 base pairs, preferably not less than 8 base pairs, 10 base pairs, 20 base pairs, 30 base pairs. Considering the difficulty of synthesis and the ease of use, the length of the nucleotide sequence should not be longer than 2000 base pairs, 1500 base pairs, 1000 base pairs, 800 base pairs, 500 base pairs, 400 base pairs, 300 base pairs, 200 base pairs, 100 base pairs.

Reagents that may interfere with the normal function of the mRNA of PAICS gene are selected from the group consisting of polypeptide hormone, enzyme, interferon, interleukin, colony stimulating factor, a nucleotide sequence that may be hybridized with PAICS gene or a modified nucleotide sequence, recombinant protein.

Reagents that may inactivate the specificity of the PAICS protein are selected from the group consisting of polypeptide hormone, enzyme, interferon, interleukin, colony stimulating factor, recombinant protein and antibody of the PAICS protein.

Method for treating human ADSL deficiency, comprising administration of preparation to a patient, said preparation comprises at least one of the following:

reagents and/or oligonucleotides that may interfere with the PAICS gene expression;
reagents and/or oligonucleotides that may interfere with the normal function of the mRNA of PAICS gene;
reagents that may inactivate the specificity of the PAICS protein.

RNA Interference with PAICS Gene can Retrieve the Phenotype with ADSL Deletion in Nematode

Many genes play crucial roles in nematode during the embryonic development and growth. Nematode is widely used in the study of human hereditary metabolic disease as its genes share about 60% homology with human [Kuwabara, P. E., and O'Neil, N. (2001). The use of functional genomics in C. elegans for studying human development and disease. Journal of Inherited Metabolic Disease 24, 127-138]. Nematode is currently used for the studies of apoptosis, neurodevelopment, behavioural biology and so on, however, no report using nematode as model organism for study of human ADSL deficiency has been found.

ADSL gene (gene NO.:R06C7.5a) mainly encodes adenylosuccinate lyase in nematode and is involved in the reaction which catalytically cleave SAICAR into AICAR and S-AMP into AMP in the metabolic pathway of de novo adenine synthesis. PAICS gene (gene NO.: B0286.3) is an upstream gene of said reaction, and mainly encodes SAICAR synthetase and is involved in the synthesis of metabolic intermediate SAICAR in the pathway of de novo adenine synthesis, thereby, it is a non-essential gene. ADSL gene is an essential gene in nematode, and when completely deleted, the embryonic development of the nematode will stop and larvae will die [Sonnichsen, B., Koski, L. B., Walsh, A., Marschall, P., Neumann, B., Brehm, M., Alleaume, A. M., Artelt, J., Bettencourt, P., Cassin, E., et al. (2005). Full-genome RNAi profiling of early embryogenesis in Caenorhabditiselegans. Nature 434, 462-469. Julian Ceron, Jean-François Rual, Abha Chandra, Denis Dupuy, Marc Vidal and Sander van den Heuvel. (2007). Large-scale RNAi screens identify novel genes that interact with the C. elegans retinoblastoma pathway as well as splicing-related components with synMuv B activity. BMC Developmental Biology 7-30]. ADSL gene of the nematode (R06C7.5a) is homologous with human ADSL gene. As a multicellular organism, the nematode is ideal for studying ADSL deficiency, and further studying impacts on phenotypes such as growth and development on nematode after ADSL is deleted.

In this study, feeding type of RNA interference was used to silence the expression of ADSL gene and PAICS gene of the nematode and observe the changes in phenotype. RT-PCR is good for validating the efficiency of RNA interference, so as to ensure the target gene has been treated with RNA interference.

Culture and Synchronization of the Nematode

(1) Culture of the Nematode

Preparation of NGM-OP50 plate: picking monoclones OP50 from LB plate and applying them to 10 ml of LB liquid culture, and culturing them overnight at 37° C. with shaking until log phase. Collecting 1 ml bacterium solution into the 1.5 ml centrifuge tube, centrifuging the solution and washing the deposit twice with M9 Buffer. After resuspended in moderate M9 Buffer, the bacterium solution was aspirated to the centre of NGM plate using a pipette, then the NGM-OP50 plate was placed in the incubator overnight at 37° C. before inoculation of nematode.

Transferring of nematode: using a sterilized spatula to cut a chunk of agar with lots of nematode from the NGM-OP50 plate and transferring them to a fresh plate, and culturing them in an incubator at 16° C. for massive culturing.

(2) Synchronization of Nematode

Using a sterilized spatula to cut a chunk of agar with adulthood worms from the NGM-OP50 plate and transferring them to a fresh plate. Excessive worms were killed with red-hot loop under the inverted microscope, leaving only one hermaphroditic nematode as F1 generation parent used to produce a large number of F2 generation larvae. After culturing in incubator at 16° C. for about 4-5 day, lots of L1-L2 stage larvae were generated. F1 generation parent was then killed with red-hot loop, and larvae were washed down from the plate by M9 Buffer. The solution was centrifuged for 2 min to wash away residual Escherichia coli OP50. Each RNAi plate was transferred with 4-5 synchronized worms.

RNAi Feeding Method for Nematode

(1) Preparation of RNAi Plate

1. HT115-L4440, HT115-L4440-1100, HT115 monoclones were picked from the LB-tetra+ plate into 2 ml of LB-Cb+ liquid culture and incubated overnight at 37° C. on a shaker.

2. The above bacterial solution was transferred with a proportion of 1:100 to 20 ml of LB-Cb+ fresh liquid culture at day 2, and incubated overnight at 37° C. with agitation until log phase (D600≈0.5), 200 μl of 0.1 M IPTG was added to obtain a final concentration of 1 mM.

3. The culture flasks were moved to 16° C. and incubated overnight with an agitation of 120 rpm.

Mix the above bacterial solution in day 3 according to below table:

| | | |
|---|---|---|
| Negative | 500 μl HT115-L4440-VAC | 500 μl HT115-L4440-VAC |
| control | | |
| RNAi | 500 μl HT115-L4440-VAC | 500 μl HT115-L4440-1100 |
| ADSL/vac | | |
| RANi | 500 μl HT115-L4440-VAC | 500 μl HT115-L4440-1600 |
| PAICS/vac | | |
| RANi | 500 μl HT115-L4440-1100 | 500 μl HT115-L4440-1600 |
| ADSL/PAICS | | |

4. The 4 mixed bacterial solutions were centrifuged at 12000 rpm for 2 min. The supernatant was removed and the centrifugated deposit was washed twice with 1 ml of M9 Buffer. The bacterial solution was resuspended with M9 buffer. The bacterial solution was dropped to the center of the NGM-IPTG-Cb+ plate which was then placed in the constant incubator and incubated overnight at 16° C.
(2) Transferring of the Synchronized F2 Generation Worms to RNAi Plate
1) 4-5 synchronized worms were transferred to RNAi plate which was then placed in the constant incubator and incubated at 16° C. for 4-5 days. It can be observed that F2 generation developed to mature individuals and began to lay eggs and produce larvae (F3 generation).

2) F3 generation larvae (with a number of about 10-20) were transferred to the corresponding RNAi plate for further observation and analysis of developmental phenotype.

3) the remaining F3 generation worms were continued to be cultured for 5-6 days on the F2 generation plate, then washed down from the plate by M9 Buffer, divided into two equal parts following by washing them twice with M9 buffer, one for RNA extraction and RT-PCR identification, another for analysis of the extraction of metabolite later, The worm was placed in a −80° C. freezer after flash freeze with liquid nitrogen.

(3) Phenotype Observation of the Growth and Development of F3 Generation Larvae

Phenotype observation of the growth and development was carried out on the F3 generation larvae which was newly growing on the RNAi plate. The worms of day 3 and day 6 (adulthood) were photographed using inverted microscope, observing the growth phenotype and measuring the size of the worm to obtain the data of the phenotype of growth and development after RNA silence.

Validate the Efficiency of RNA Interference by Using RT-PCR

(1) Extraction of the Nematode RNA (TRIzol Reagent Instruction, Invitrogen)

The worms were washed down from the plate by using M9 Buffer, collected in 1.5 ml centrifuge tube, and centrifuged at 400 g for 2 min and the residual Escherichia coli was removed via washing twice with M9 Buffer.

Collected worms were quick freezed using liquid nitrogen. 1 ml of TRIzol Reagent and liquid nitrogen were added, then the worms were grinded using grinding rod to powder form.

After standing for 5 min at room temperature, 0.2 ml of chloroform was added and followed by tightening the lid securely, turning upside down to mix for 16 s and letting it stand for 2-3 min, then it was centrifuged at 12000 g at 4° C. for 15 mins.

The colorless supernatant (about 0.5 ml) was aspirated to a new 1.5 ml centrifuge tube. After the addition of 0.5 ml of isoamyl alcohol, the tube was turned upside down to mix, and then left standing for 10 min.

Centrifuging the tube at 12000 g at 4° C. for 10 min, it can be seen the white flocculent sediment.

The supernatant was tipped off carefully and 1 ml of 75% anhydrous ethanol (diluted in DEPC water was added.

After centrifugation at 12000 g at 4° C. for 5 min and tipping off the supernatant carefully, the RNA sediment was dried at room temperature for 10 min.

Certain amount of RNA free water was added to dissolve the RNA sediment and the RNA extraction result was determined by electrophoresis on gel containing formaldehyde.

(2) RNA was Reversely Transcribed into cDNA (PrimerScript RT Reagent Kit, Takara)

Reverse transcription reaction system:

| Reagents | Volume (μl) | |
|---|---|---|
| 5× PrimerScript Nuffer | 4 | μl |
| PrimerScript RT Enzyme Kit I | 1 | μl |
| OligodT Primer | 1 | μl |
| Random 6 mers | 1 | μl |
| Total RNA | ≦500 | ng |

| | |
|---|---|
| RNase Free H₂O | Up to 20 μl |

The reverse transcription reaction conditions are as follows: 37° C., 45 min; 85° C., 5 s.

(3) The RT-PCR Reaction System (SYBR Premix Ex Taq II, Takara)

The RT-PCR was carried out by adding corresponding reagents according to the instruction of SYBR Premix Ex Taq II.

| Reagents | Volume | |
|---|---|---|
| SYBR Premix | 5 | μl |
| Sence Primer | 0.4 | μl |
| Anti-sense primer | 0.4 | μl |
| cDNA | 1 | μl |

| | |
|---|---|
| H₂O | Up to 10 μl |

Primer sequences for RT-PCR:

| Primers | Sequences | SEQ ID NO: |
|---|---|---|
| RT-PAICS-S | ACAGTCTTATCGGGACCTCAAA | 1 |
| RT-PAICS-A | AGAGCCCATCAACACTACAACC | 2 |
| RT-ADSL-S | CACCTTGGTGCTACTTCTTGCT | 3 |
| RT-ADSL-A | GGGTAGACTGGCTCGTTCCTT | 4 |
| RT-actin-S | ACCGAGCGTGGTTACTCTTTCA | 5 |
| RT-actin-A | TCCGACGGTGATGACTTGTCC | 6 |

Biological repetition were done three times on each sample.

RT-PCR procedure setting (two-step method):

Pre-denaturation: 95° C. 30 s, 1 cycle.

PCR reaction: 95° C. 10 s, 60° C. 20 s, 40 cycles.

(4) RT-PCR Data Analysis

The internal reference of the experiment is the β-actin gene of the nematode which is used to measure the relative expression of ADSL and PAICS. Data analysis applies common Ct value comparison method 2^{-ΔΔCT} (formula see below). The result is the fold change of the expression of the target gene in experimental group with respect to the internal reference, compared with the untreated group.

ΔΔCT=(Ct_{Target Genes}−Ct_{Internal})_{Experimental Group}−(Ct_{Target Genes}−Ct_{Internal})_{Untreated Group}

Fold change=2⁻^{_{ΔΔ}}^{CT}

(5) A Total RNA Electrophoretogram of the Nematode in F3 Generation

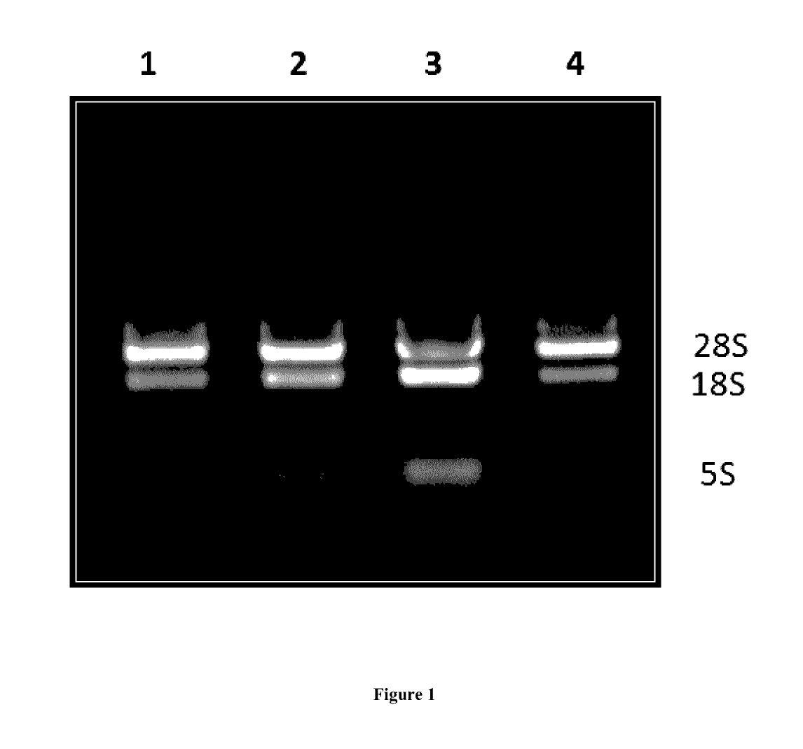

FIG. 1 shows a total RNA electrophoretogram of the nematode in F3 generation. It can be seen from the FIG. 1 that the quality of the total extracted RNA is good, and the bands are clear, in which the brightness of 28S is twice of the 18S, without showing degeneration and tailing phenomenon of RNA band. Therefore, it can be used for further RT-PCR experiment.

(6) Analysis of the Efficiency of RNA Interference with ADSL and PAICS in Nematode by RT-PCR

TABLE 1
Determination of the relative expression
of ADSL and PAICS by RT-PCR

Gene relative expression level

| Sample name | Gene PAICS | Gene ADSL |
|---|---|---|
| Negative Control | 100.00% | 100.00% |
| RNAi PAICS | 37.89% | 105.95% |
| RNAi ADSL | 132.87% | 35.60% |
| RNAi PAICS | 41.75% | 38.87% |

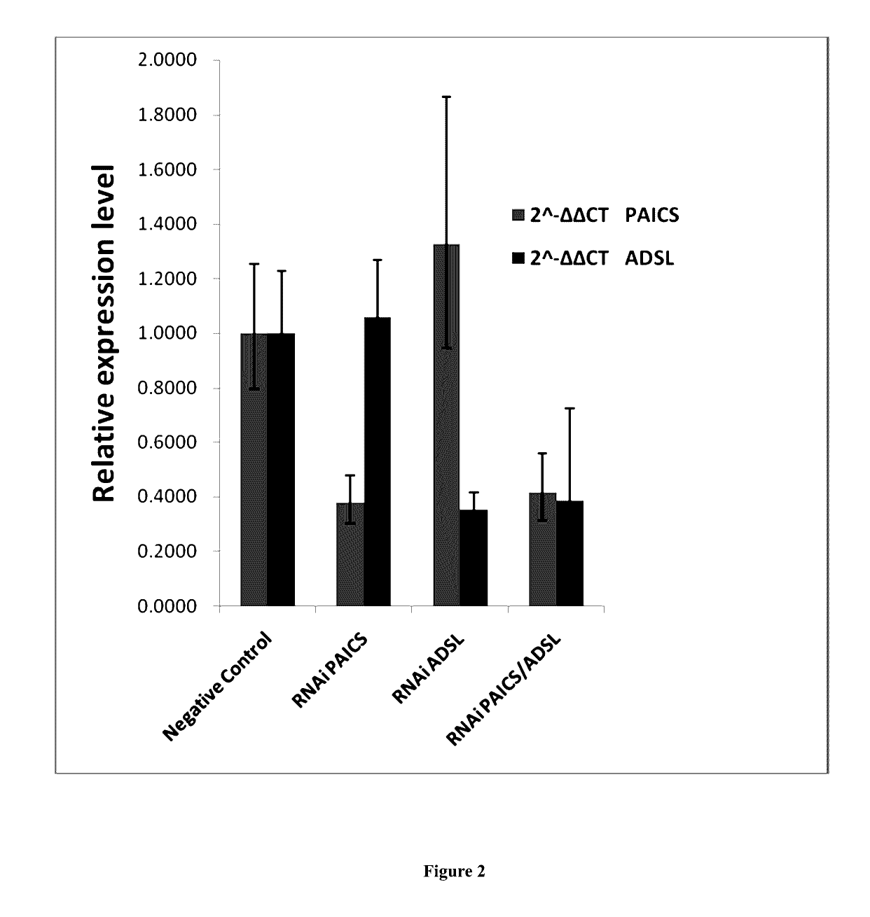

The efficiency of RNA interference in nematode can be validated by RT-PCR which is suitable for measuring the relative expression of certain target gene. In this experiment, we compared the relative expression of the target gene ADSL and PAICS in different treated samples, using house keeping gene β-actin as internal reference. FIG. 2 shows the relative expression of PAICS gene and ADSL gene in different treatment groups. During the process of RT-PCR, the expression of the gene was calculated by Cp value which is proportional to the initial copy number of the template. In the case of definite concentration of cDNA, the initial concentration of certain template is linear with the Cp value.

It can be found from the table 1 that when the PAICS gene was solely treated with RNA interference, the expression of PAICS is only 37.89% of the negative control in nematode, but no change occurs in that of ADSL. That is, sole interference with PAICS will not affect the expression of ADSL gene. When the ADSL gene was solely treated with RNA interference, the expression of ADSL is only 35.6% of the negative control in nematode, the silencing efficiency of gene expression is about 64.4%, while the expression of PAICS was up-regulated. When the ADSL and the PAICS gene are both treated with RNA interference, the expression of ADSL and PAICS are 38.87% and 41.75% of the negative control respectively in nematode.

The results of table 1 and FIG. 2 have shown that the efficiency of RNA interference with ADSL and PAICS in nematode is high and consistent with expectant experiment data, and the next observation of growth and development phenotype and analysis of metabolite can be done.

(7) Phenotype Observation on Nematode with RNA Interference with Different Genes

Here in before, RT-PCR has validated the feasibility of RNA interference with the expression of certain gene in nematode. Through the observation of the growth and development of F3 generation larvae, it can be analyzed the corresponding phenotype changes in nematode after RNA interference, thereby investigating the effect of certain gene on the growth and development in nematode, and its function in cells.

Through the phenotype observation of F3 generation, the change of growth and development and the spawning number after RNA interference with a specific gene can be reflected more truly due to no significant change of phenotype of F2 generation larvae was observed on the RNA interference treated plate. Nematodes turn into adults in about 6 days in constant incubator at 16° C., and phenotype observation on F3 generation larvae will carried out at day 3 and day 6. The worms (≧10 worms for each group) were photographed using upright microscope, and the lengths of the worms were measured using Image J to obtain the raw data of growth and development of F3 generation larvae.

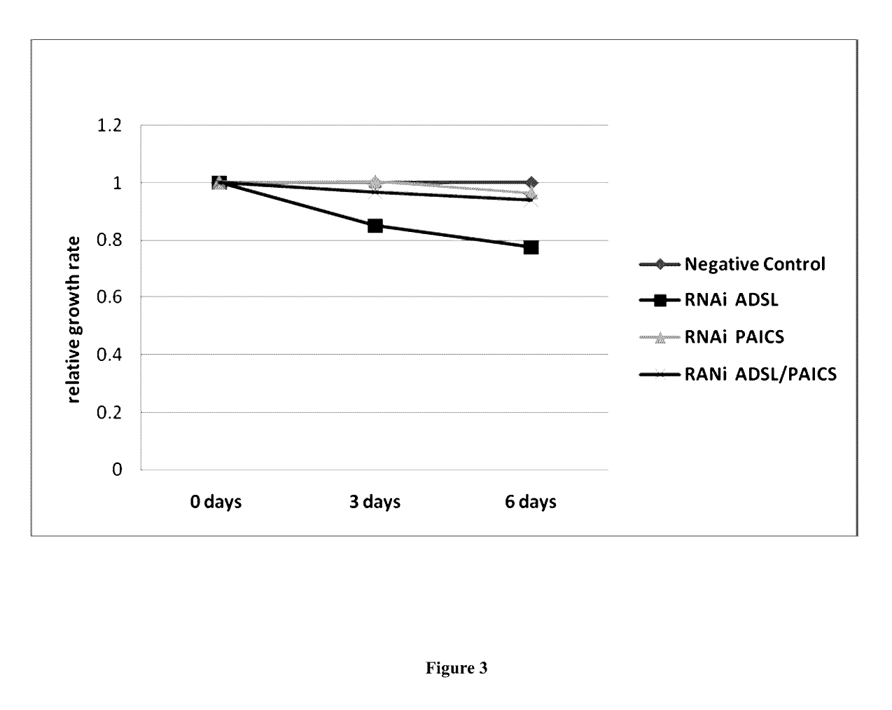

FIG. 3 shows the relative growth rate of the nematode after RNA interference (0 day, 3 days, 6 days). The result shows that the sole RNA interference with ADSL gene in nematode would lead to inhibition of the growth and development of the nematode, and nematode whose PAICS gene is solely treated with RNA interference is normal in the growth and development, which is similar to the growth rate of the negative control. While the ADSL and PAICS gene of the nematode are both treated with RNA interference, the F3 generation larvae exhibits normal growth and development with a growth rate substantially the same as negative control.

(8) RNA Interference with PAICS Gene in Nematode can Retrieve the Lethal Phenotype with ADSL Deficiency

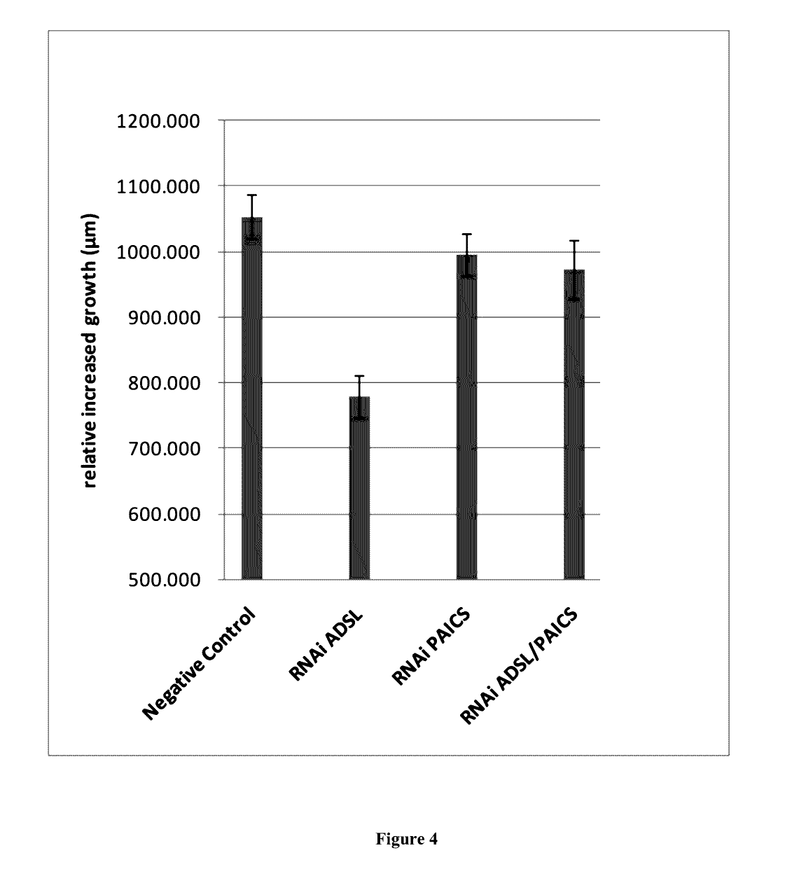

FIG. 4 shows the relative increase in length of the nematode after interference. A significant change in relative growth length of nematode can be found among different groups. The growth and development of nematode whose PAICS gene is solely treated with RNA interference is slower than that of negative control, while the growth and development inhibition caused by ADSL deficiency can be relieved when the ADSL and PAICS gene of the nematode are both treated with RNA interference.

TABLE 2
The relative growth rate of the F3 generation
nematode after RNA interference

| Sample | growth rate |
|---|---|
| Negative Control | 100% |
| RNAi ADSL | 73.94% |
| RNAi PAICS | 94.51% |
| RNAi ADSL/PAICS | 92.40% |

The experimental result can be analyzed more directly through the data in table 2. PAICS gene is a non essential gene in nematode, and no significant change will be shown in phenotype when deleted or mutated. The F3 generation nematode whose PAICS gene is solely treated with RNA interference is normal in the growth and development, without showing the phenotype such as lagged development, sluggish in motion, etc. (the growth rate can reach 94.51% of the negative control).

After sole RNA interference with ADSL gene in F3 generation nematode, they grow slowly (only 73.94% of the negative control) and exhibit sluggish in motion, which is consistent with the previous report, wherein ADSL is an essential gene which will cause severe growth and development disorder in nematode when deleted or mutated [Sonnichsen, B., Koski, L. B., Walsh, A., Marschall, P., Neumann, B., Brehm, M., Alleaume, A. M., Artelt, J., Bettencourt, P., Cassin, E., et al. (2005). Full-genome RNAi profiling of early embryogenesis in Caenorhabditiselegans. Nature 434, 462-469. Julian Ceron, Jean-François Rual, Abha Chandra, Denis Dupuy, Marc Vidal and Sander van den Heuvel. (2007). Large-scale RNAi screens identify novel genes that interact with the C. elegans retinoblastoma pathway as well as splicing-related components with synMuv B activity. BMC Developmental Biology 7-30]. Furthermore, the spawning number in F2 generation whose ADSL gene is solely treated with RNA interference is much less than the negative control (not shown).

RNA interference with PAICS in nematode can retrieve severe phenotype with ADSL deficiency, and the growth and development of F3 generation nematode tends to be normal, reaching the rate similar to the negative control (such as reaching 92.40% of that of the negative control).

1. A composition for the treatment of adenylosuccinatelyase deficiency, comprising a reagent or an oligonucleotide which interferes with the expression of the gene encoding phosphoribosylaminoimidazolesuccinocarboxamidesynthetase/phosphoribosylaminoimidazole carboxylase (PAICS), and/or interferes with the normal function of mRNA of the gene encoding PAICS and/or inactivates the specificity of the PAICS protein.

2. The composition of claim 1, wherein the reagent is chosen from the group consisting of polypeptide hormone, enzyme, interferon, interleukin, colony stimulating factor, a nucleotide sequence that hybridizes with PAICS gene, a modified nucleotide sequence, recombinant protein, an antibody of the PAICS protein and mixtures thereof.

3. The composition of claim 1, wherein the oligonucleotide comprises a nucleotide sequence that hybridizes with the cDNA sequence of the human PAICS gene.

4. The composition of claim 1, wherein the oligonucleotide is from about 5 to 2000 base pairs.

5. The composition of claim 1, wherein the reagent is an interfering RNA.

6. A method for the treatment of adenylosuccinatelyase deficiency in a subject in need thereof, comprising administering to the subject a therapeutically effective amount of a reagent or a oligonucleotide which interferes with the expression of the gene encoding phosphoribosylaminoimidazolesuccinocarboxamidesynthetase/phosphoribosylaminoimidazole carboxylase (PAICS), and/or interferes with the normal function of mRNA of the gene encoding PAICS and/or inactivates the specificity of the PAICS protein.

7. The method of claim 6, wherein the subject is human.

8. The method of claim 6, wherein the reagent is chosen from the group consisting of polypeptide hormone, enzyme, interferon, interleukin, colony stimulating factor, a nucleotide sequence that hybridizes with PAICS gene, a modified nucleotide sequence, recombinant protein, an antibody of the PAICS protein and mixtures thereof.

9. The method of claim 6, wherein the oligonucleotide comprises a nucleotide sequence that hybridizes with the cDNA sequence of the human PAICS gene.

10. The method of claim 6, wherein the oligonucleotide is from about 5 to 2000 base pairs.

11. The method of claim 6, wherein the reagent is an interfering RNA.

12. A method for relieving the symptoms of adenylosuccinatelyase deficiency in a subject in need thereof, comprising administering to the subject a therapeutically effective amount of a reagent or a oligonucleotide which interferes with the expression of the gene encoding phosphoribosylaminoimidazolesuccinocarboxamidesynthetase/phosphoribosylaminoimidazole carboxylase (PAICS), and/or interferes with the normal function of mRNA of the gene encoding PAICS and/or inactivates the specificity of the PAICS protein.

13. The method of claim 12, wherein the subject is human.

14. The method of claim 12, wherein the reagent is chosen from the group consisting of polypeptide hormone, enzyme, interferon, interleukin, colony stimulating factor, a nucleotide sequence that hybridizes with PAICS gene, a modified nucleotide sequence, recombinant protein, an antibody of the PAICS protein and mixtures thereof.

15. The method of claim 12, wherein the oligonucleotide comprises a nucleotide sequence that hybridizes with the cDNA sequence of the human PAICS gene.

16. The method of claim 12, wherein the oligonucleotide is from about 5 to 2000 base pairs.

17. The method of claim 12, wherein the reagent is an interfering RNA.

18. The method of claim 12, wherein the symptoms are the accumulation of excessive accumulation of enzyme substrate SAICAR in bodily fluid and cells, and cell damage.

19. A method of identifying the composition of claim 1, comprising:
a. measuring the level of expression of the gene encoding phosphoribosylaminoimidazolesuccinocarboxamidesynthetase/phosphoribosylaminoimidazole carboxylase (PAICS);
b. contacting a regent or an oligonucleotide with the gene encoding PAICS;
c. measuring the level of expression of the gene encoding PAICS after contact with the reagent or oligonucleotide;
d. comparing the level of expression obtained in step c) with the level of expression in step a); and
e. determining that if the level of expression in step c) is less than the level of expression in step a), the reagent or oligonucleotide is identified as a composition for the treatment of adenylosuccinatelyase deficiency.

20. The method of claim 19, wherein the level of gene expression is measured by the level of DNA or mRNA.

21. The method of claim 19, wherein the level of gene expression is measured using polymerase chain reaction.

22. The method of claim 19, wherein the gene encoding PAICS is contained within a host cell or animal.

23. The method of claim 22, wherein the animal is a nematode.

24. A method of identifying the composition of claim 1, comprising:
a. measuring or observing a phenotype in a host cell or animal possessing the gene encoding phosphoribosylaminoimidazolesuccinocarboxamidesynthetase/phosphoribosylaminoimidazole carboxylase (PAICS);
b. contacting a reagent with the host cell or animal;
c. measuring or observing the phenotype in the host cell or animal after contacting the reagent with the host cell or animal;
d. comparing the measurement or observation in step a) with the measurement or observation in step c); and
e. determining that if the measurement or observation in step a) is different than the measurement or observation in step c), the reagent is identified as a composition for the treatment of adenylosuccinatelyase deficiency.

25. The method of claim 24, wherein the animal is a nematode.

26. The method of claim 24, wherein the phenotype that is observed or measured is the excessive accumulation of enzyme substrate SAICAR in bodily fluid and cells, and cell damage, and a decrease in the phenotype in the host cell or animal after contact with the reagent identifies the reagent as a composition for the treatment of adenylosuccinatelyase deficiency.

27. The method of claim 24, wherein the phenotype that is observed or measured is growth of the animal, and an increase in the phenotype in the animal after contact with the reagent identifies the reagent as a composition for the treatment of adenylosuccinatelyase deficiency.

28. A method of identifying the composition of claim 1, comprising:
a. contacting a regent or an oligonucleotide with the gene encoding phosphoribosylaminoimidazolesuccinocarboxamidesynthetase/phosphoribosylaminoimidazole carboxylase (PAICS); and
b. determining if the reagent or oligonucleotide binds to the gene encoding PAICS, wherein if the reagent or oligonucleotide binds to the gene encoding PAICS, the reagent or oligonucleotide is identified as a composition for the treatment of adenylosuccinatelyase deficiency.

29. A method of identifying the composition of claim 1, comprising:
a. contacting a reagent or an oligonucleotide with the phosphoribosylaminoimidazolesuccinocarboxamidesynthetase/phosphoribosylaminoimidazole carboxylase (PAICS) protein; and
b. detecting the presence of a complex between the reagent or oligonucleotide and the protein,
wherein the presence of the complex between the reagent or oligonucleotide and the protein identifies the reagent or oligonucleotide as a composition for the treatment of adenylosuccinatelyase deficiency.
